(19) United States
(12) Patent Application Publication
(10) Pub. No.: US 2022/0403372 A1
(43) Pub. Date: Dec. 22, 2022

(54) METHOD FOR EXTRACTION OF CELL-FREE DNA

(71) Applicant: LUMINEX CORPORATION, Austin, TX (US)

(72) Inventor: Steven KRUEGER, Austin, TX (US)

(73) Assignee: LUMINEX CORPORATION, Austin, TX (US)

(21) Appl. No.: 17/838,767

(22) Filed: Jun. 13, 2022

Related U.S. Application Data
(60) Provisional application No. 63/212,274, filed on Jun. 18, 2021.

(51) Int. Cl. C12N 15/10

(52) U.S. Cl. CPC C12N 15/1006

(57) ABSTRACT

Provided herein are compositions and methods for extracting cell-free DNA (cfDNA) from a biological sample without the need for pretreatment with proteases prior to binding of the cfDNA to a solid substrate and without a heated elution step. The methods comprise contacting the biological sample with a chelating agent and an oxidizing agent prior to eluting the cell-free DNA from a solid substrate.

### REFERENCE TO RELATED APPLICATIONS

The present application claims the priority benefit of U.S. provisional application No. 63/212,274, filed Jun. 18, 2021, the entire contents of which is incorporated herein by reference.

### BACKGROUND

### 1. Field

The present disclosure relates generally to the field of molecular biology. More particularly, it concerns compositions and methods for extracting cell-free DNA from plasma.

### 2. Description of Related Art

Methods for extraction of nucleic acids from biological samples are known. One commonly used method involves the addition of a chaotropic agent to a complex biological sample to form a lysate, followed by binding of nucleic acids to a solid silica substrate (see U.S. Pat. No. 5,234,809). Typically, after binding of the nucleic acids to the silica substrate and removal of the non-bound components, one or more washes are performed, followed by elution of the nucleic acids from the substrate. The silica substrate may be provided in the form of a packed column (stationary solid phase) or in the form of particles (mobile solid phase) which typically have magnetic or paramagnetic cores to facilitate separation from the non-binding components of the lysate.

Efficient isolation of nucleic acids from blood products using the aforementioned methods are known to be challenging. In particular, blood products tend to plug stationary solid phase systems, preventing the lysate from passing through the solid phase. Use of blood products with mobile solid phase systems generally results in poor yields of nucleic acids. To overcome this problem, blood product lysates are typically treated with proteinase K to remove unwanted protein prior to addition to silica substrates. However, such treatments add to the cost and complexity of nucleic acid extraction, as they often require prolonged heat treatment and separate addition of proteinase K and chaotropic agents. Methods that provide for efficient extraction of nucleic acids from blood products and that do not require the use of proteinase K are needed.

### SUMMARY

Provided herein are compositions and methods for extracting cell-free DNA (cfDNA) from biological samples, such as blood plasma samples, by contacting the biological sample with a chelating agent and an oxidizing agent prior to eluting the cell-free DNA from a solid substrate. The methods provide for extraction of cfDNA without a protease treatment step. The methods also provide for extraction of cfDNA without a heated elution step.

In one embodiment, the methods comprise: (1) combining the biological sample with a solid substrate and a binding buffer comprising a chaotropic agent in order to bind cfDNA from the biological sample to the solid substrate, wherein the biological sample has not been treated with proteinase K; (2) washing the cfDNA bound to the solid substrate with a wash buffer; and (3) eluting the cfDNA from the solid substrate, wherein the binding buffer and/or the wash buffer comprises a chelating agent and an oxidizing agent.

In some aspects, the binding buffer comprises a chaotropic agent, a chelating agent, and an oxidizing agent. In some aspects, the wash buffer comprises a chelating agent and an oxidizing agent.

In various aspects, binding cfDNA from the biological sample to the solid substrate is performed by incubating the combined biological sample, solid substrate, and binding buffer at ambient temperature. The incubation may be for about 5 minutes, about or at least 6 minutes, about or at least 7 minutes, about or at least 8 minutes, about or at least 9 minutes, or about or at least 10 minutes. The incubation may be for at most 10 minutes, at most 9 minutes, at most 8 minutes, at most 7 minutes, at most 6 minutes, or at most 5 minutes.

In various aspects, the methods do not include a heat treatment step. Heat treatment steps include incubating the biological sample, or any solution used during during the course of performing the methods, at a temperature above ambient temperature. For example, a heat treatment step could include incubation at a temperature that is between about 35° C. and about 80° C. (e.g., about or at least 35° C., 40° C., 45° C., 50° C., 55° C., 60° C., 65° C., 70° C., 75° C., or 80° C.) for at least 10 seconds. Specific examples of heat treatment steps include incubation at 60° C. for 30 minutes and incubation at 70° C. for 10 minutes.

In various aspects, eluting is performed at ambient temperature. In various aspects, eluting is performed for about or at least 15 seconds, about or at least 20 seconds, about or at least 25 seconds, about or at least 30 seconds, about or at least 35 seconds, about or at least 40 seconds, about or at least 45 seconds, about or at least 50 seconds, about or at least 55 seconds or about or at least 60 seconds. In various aspects, eluting is complete with in at most 30 seconds, at most 45 seconds, at most 60 seconds, at most 2 minutes, at most 3 minutes, at most 4 minutes, or at most 5 minutes.

The chelating agent may be an amine-based chelating agent (e.g., EDTA or MGDA) or a phosphate-based chelating agent (e.g., STP). The concentration of the chelating agent (e.g., EDTA, MGDA, or STP) in the binding buffer and/or the wash buffer may be between about 5 mM and about 100 mM, between about 10 mM and about 100 mM, between about 15 mM and about 100 mM, between about 20 mM and about 100 mM, between about 25 mM and about 100 mM, between about 30 mM and about 100 mM, between about 35 mM and about 100 mM, between about 40 mM and about 100 mM, between about 45 mM and about 100 mM, between about 50 mM and about 100 mM, or any range derivable therein. The concentration of the chelating agent (e.g., EDTA, MGDA, or STP) in the binding buffer and/or the wash buffer may be at least 5 mM, at least 6 mM, at least 7 mM, at least 8 mM, at least 9 mM, at least 10 mM, at least 11 mM, at least 12 mM, at least 14 mM, at least 16 mM, at least 18 mM, at least 20 mM, at least 22 mM, at least 24 mM, at least 25 mM, at least 26 mM, at least 28 mM, at least 30 mM, at least 32 mM, at least 34 mM, at least 36 mM, at least 38 mM, at least 40 mM, at least 42 mM, at least 44 mM, at least 46 mM, at least 48 mM, at least 50 mM, at least 52 mM, at least 54 mM, at least 56 mM, at least 58 mM, at least 60 mM, at least 62 mM, at least 64 mM, at least 66 mM, at least 68 mM, at least 70 mM, at least 72 mM, or at least 74 mM. The concentration of the chelating agent (e.g., EDTA, MGDA, or STP) in the binding buffer and/or the wash buffer may be about 5 mM, about 6 mM, about 7 mM, about 8 mM, about 9 mM, about 10 mM, about 11 mM, about 12 mM, about 14 mM, about 16 mM, about 18 mM, about 20 mM, about 22 mM, about 24 mM, about 25 mM, about 26 mM, about 28 mM, about 30 mM, about 32 mM, about 34 mM, about 36 mM, about 38 mM, about 40 mM, about 42 mM, about 44 mM, about 46 mM, about 48 mM, about 50 mM, about 52 mM, about 54 mM, about 56 mM, about 58 mM, about 60 mM, about 62 mM, about 64 mM, about 66 mM, about 68 mM, about 70 mM, about 72 mM, or about 74 mM.

The oxidizing agent may be sodium percarbonate or hydrogen peroxide. The concentration of the oxidizing agent (e.g., sodium percarbonate or hydrogen peroxide) in the binding buffer and/or wash buffer may be between about 1 mM and about 100 mM, between about 5 mM and about 100 mM, between about 10 mM and about 100 mM, between about 15 mM and about 100 mM, between about 20 mM and about 100 mM, between about 25 mM and about 100 mM, between about 30 mM and about 100 mM, between about 35 mM and about 100 mM, between about 40 mM and about 100 mM, between about 45 mM and about 100 mM, between about 50 mM and about 100 mM, or any range derivable therein. The concentration of the oxidizing agent (e.g., sodium percarbonate or hydrogen peroxide) in the binding buffer and/or wash buffer may be at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 14 mM, at least 16 mM, at least 18 mM, at least 20 mM, at least 22 mM, at least 24 mM, at least 26 mM, at least 28 mM, at least 30 mM, at least 32 mM, at least 34 mM, at least 36 mM, at least 38 mM, at least 40 mM, at least 42 mM, at least 44 mM, at least 46 mM, at least 48 mM, at least 50 mM, at least 52 mM, at least 54 mM, at least 56 mM, at least 58 mM, at least 60 mM, at least 62 mM, or at least 64 mM. The concentration of the oxidizing agent (e.g., sodium percarbonate or hydrogen peroxide) in the binding buffer and/or the wash buffer may be about 1 mM, about 2 mM, about 3 mM, about 4 mM, about 5 mM, about 6 mM, about 7 mM, about 8 mM, about 9 mM, about 10 mM, about 11 mM, about 12 mM, about 14 mM, about 16 mM, about 18 mM, about 20 mM, about 22 mM, about 24 mM, about 26 mM, about 28 mM, about 30 mM, about 32 mM, about 34 mM, about 36 mM, about 38 mM, about 40 mM, about 42 mM, about 44 mM, about 46 mM, about 48 mM, about 50 mM, about 52 mM, about 54 mM, about 56 mM, about 58 mM, about 60 mM, about 62 mM, or about 64 mM.

In some aspects, when the chelating agent is EDTA or MGDA, the oxidizing agent is sodium percarbonate. In some aspects, when the chelating agent is STP, the oxidizing agent is sodium percarbonate or hydrogen peroxide.

In some aspects, the binding buffer comprises an alcohol, such as, for example, isopropyl alcohol. In some aspects, the wash buffer comprises an alcohol, such as, for example, isopropyl alcohol. The isopropyl alcohol may make up between about 15% and about 60%, between about 20% and about 60%, between about 25% and about 60%, between about 30% and about 60%, between about 35% and about 60%, between about 40% and about 60%, between about 45% and about 60% by volume, or any range derivable therein, of the binding buffer and/or the wash buffer. The isopropyl alcohol may make up at least 15%, at least 16%, at least 17%, at least 18%, at least 19%, at least 20%, at least 22%, at least 24%, at least 26%, at least 28%, at least 30%, at least 32%, at least 34%, at least 36%, at least 38%, at least 40%, at least 42%, at least 44%, at least 46%, at least 48%, or at least 50% by volume of the binding buffer and/or the wash buffer. The isopropyl alcohol may make up about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, or about 50% by volume of the binding buffer and/or the wash buffer.

In some aspects, the solid substrate is a silica substrate, such as silica beads, silica-coated beads, a silica column, or a silica membrane. The silica-coated beads may be magnetic or paramagnetic.

In various aspects, the chaotropic agent in the binding buffer is a guanidine salt, such as, for example, guanidine hydrochloride, guanidine thiocyanate, or guanidine isothiocyanate. The concentration of guanidine in the binding buffer may be between about 1 M and about 10 M, between about 2 M and about 10 M, between about 3 M and about 10 M, between about 4 M and about 10 M, between about 5 M and about 10 M, between about 6 M and about 10 M, between about 7 M and about 10 M, between about 8 M and about 10 M, or any range derivable therein. The concentration of guanidine in the binding buffer may be at least 1 M, at least 2 M, at least 3, M, at least 4 M, at least 5 M, at least 6 M, at least 7 M, at least 8 M, at least 9 M, or at least 10 M. The concentration of guanidine in the binding buffer may be about 1 M, about 1.5 M, about 2 M, about 2.5 M, about 3 M, about 3.4 M, about 3.5 M, about 4 M, about 4.5M, about 5 M, about 6 M, about 7 M, about 8 M, about 9 M, or about 10 M.

In one embodiment, provided herein are compositions for use in extracting cell-free DNA from plasma. The compositions may comprise an oxidizing agent, a chelating agent, and a chaotropic agent.

The chelating agent may be EDTA, MGDA, or STP. The concentration of the chelating agent (e.g., EDTA, MGDA, or STP) in the composition may be between about 5 mM and about 100 mM, between about 10 mM and about 100 mM, between about 15 mM and about 100 mM, between about 20 mM and about 100 mM, between about 25 mM and about 100 mM, between about 30 mM and about 100 mM, between about 35 mM and about 100 mM, between about 40 mM and about 100 mM, between about 45 mM and about 100 mM, between about 50 mM and about 100 mM, or any range derivable therein. The concentration of the chelating agent (e.g., EDTA, MGDA, or STP) in the composition may be at least 5 mM, at least 6 mM, at least 7 mM, at least 8 mM, at least 9 mM, at least 10 mM, at least 11 mM, at least 12 mM, at least 14 mM, at least 16 mM, at least 18 mM, at least 20 mM, at least 22 mM, at least 24 mM, at least 26 mM, at least 28 mM, at least 30 mM, at least 32 mM, at least 34 mM, at least 36 mM, at least 38 mM, at least 40 mM, at least 42 mM, at least 44 mM, at least 46 mM, at least 48 mM, at least 50 mM, at least 52 mM, at least 54 mM, at least 56 mM, at least 58 mM, at least 60 mM, at least 62 mM, at least 64 mM, at least 66 mM, at least 68 mM, at least 70 mM, at least 72 mM, or at least 74 mM. The concentration of the chelating agent (e.g., EDTA, MGDA, or STP) in the composition may be about 5 mM, about 6 mM, about 7 mM, about 8 mM, about 9 mM, about 10 mM, about 11 mM, about 12 mM, about 14 mM, about 16 mM, about 18 mM, about 20 mM, about 22 mM, about 24 mM, about 26 mM, about 28 mM, about 30 mM, about 32 mM, about 34 mM, about 36 mM, about 38 mM, about 40 mM, about 42 mM, about 44 mM, about 46 mM, about 48 mM, about 50 mM, about 52 mM, about 54 mM, about 56 mM, about 58 mM, about 60 mM, about 62 mM, about 64 mM, about 66 mM, about 68 mM, about 70 mM, about 72 mM, or about 74 mM.

In one aspect, the composition comprises at least or about 20 mM EDTA. In one aspect, the composition comprises at least or about 11 mM MGDA. In one aspect, the composition comprises at least or about 14 mM STP.

The oxidizing agent may be sodium percarbonate or hydrogen peroxide. The concentration of the oxidizing agent (e.g., sodium percarbonate or hydrogen peroxide) in the composition may be between about 1 mM and about 100 mM, between about 5 mM and about 100 mM, between about 10 mM and about 100 mM, between about 15 mM and about 100 mM, between about 20 mM and about 100 mM, between about 25 mM and about 100 mM, between about 30 mM and about 100 mM, between about 35 mM and about 100 mM, between about 40 mM and about 100 mM, between about 45 mM and about 100 mM, between about 50 mM and about 100 mM, or any range derivable therein. The concentration of the oxidizing agent (e.g., sodium percarbonate or hydrogen peroxide) in the composition may be at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 14 mM, at least 16 mM, at least 18 mM, at least 20 mM, at least 22 mM, at least 24 mM, at least 26 mM, at least 28 mM, at least 30 mM, at least 32 mM, at least 34 mM, at least 36 mM, at least 38 mM, at least 40 mM, at least 42 mM, at least 44 mM, at least 46 mM, at least 48 mM, at least 50 mM, at least 52 mM, at least 54 mM, at least 56 mM, at least 58 mM, at least 60 mM, at least 62 mM, or at least 64 mM. The concentration of the oxidizing agent (e.g., sodium percarbonate or hydrogen peroxide) in the composition may be about 1 mM, about 2 mM, about 3 mM, about 4 mM, about 5 mM, about 6 mM, about 7 mM, about 8 mM, about 9 mM, about 10 mM, about 11 mM, about 12 mM, about 14 mM, about 16 mM, about 18 mM, about 20 mM, about 22 mM, about 24 mM, about 26 mM, about 28 mM, about 30 mM, about 32 mM, about 34 mM, about 36 mM, about 38 mM, about 40 mM, about 42 mM, about 44 mM, about 46 mM, about 48 mM, about 50 mM, about 52 mM, about 54 mM, about 56 mM, about 58 mM, about 60 mM, about 62 mM, or about 64 mM.

In one aspect, the composition comprises at least or about 5 mM sodium percarbonate. In one aspect, the composition comprises at least or about 5 mM hydrogen peroxide.

In some aspects, when the chelating agent is EDTA or MGDA, the oxidizing agent is sodium percarbonate. In some aspects, when the chelating agent is STP, the oxidizing agent is sodium percarbonate or hydrogen peroxide.

The composition may further comprise an alcohol, such as, for example, isopropyl alcohol. The composition may comprise at least 20% isopropyl alcohol. The isopropyl alcohol may make up between about 15% and about 60%, between about 20% and about 60%, between about 25% and about 60%, between about 30% and about 60%, between about 35% and about 60%, between about 40% and about 60%, between about 45% and about 60% by volume, or any range derivable therein, of the composition. The isopropyl alcohol may make up at least 15%, at least 16%, at least 17%, at least 18%, at least 19%, at least 20%, at least 22%, at least 24%, at least 26%, at least 28%, at least 30%, at least 32%, at least 34%, at least 36%, at least 38%, at least 40%, at least 42%, at least 44%, at least 46%, at least 48%, or at least 50% by volume of the composition. The isopropyl alcohol may make up about 15%, about 16%, about 17%, about 18%, about 19%, about 20%, about 22%, about 24%, about 26%, about 28%, about 30%, about 32%, about 34%, about 36%, about 38%, about 40%, about 42%, about 44%, about 46%, about 48%, or about 50% by volume of the composition.

The chaotropic agent may be a guanidine salt, such as, for example, guanidine hydrochloride, guanidine thiocyanate, or guanidine isothiocyanate. The concentration of guanidine in the composition may be between about 0.1 M and about 10 M, between about 0.5 M and about 10 M, between about 1 M and about 10 M, between about 2 M and about 10 M, between about 3 M and about 10 M, between about 4 M and about 10 M, between about 5 M and about 10 M, between about 6 M and about 10 M, between about 7 M and about 10 M, between about 8 M and about 10 M, or any range derivable therein. The concentration of guanidine in the composition may be at least 0.1 M, at least 0.5 M, at least 1 M, at least 2 M, at least 3, M, at least 4 M, at least 5 M, at least 6 M, at least 7 M, at least 8 M, at least 9 M, or at least 10 M. The concentration of guanidine in the composition may be about 0.1. M, about 0.5 M, about 1 M, about 1.5 M, about 2 M, about 2.5 M, about 3 M, about 3.4 M, about 3.5 M, about 4 M, about 4.5M, about 5 M, about 6 M, about 7 M, about 8 M, about 9 M, or about 10 M.

Any of the binding buffer, the wash buffers, or the compositions may comprise a buffering agent. Exemplary buffering agents include, for example, Tris, TRIZMA®, Tricine, HEPES, and MOPS. Any of the binding buffers, the wash buffers, or the compositions may have a pH between 7 and 9, where the pH is measured at ambient temperature. The pH may be between 7 and 7.5, 7 and 8, 7 and 8.5, 7.5 and 8, 7.5 and 8.5, 7.5 and 9, 8 and 8.5, 8 and 9, or 8.5 and 9, or any range derivable therein. The pH may be at least 7.0, at least 7.1, at least 7.2, at least 7.3, at least 7.4, at least 7.5, at least 7.6, at least 7.7, at least 7.8, at least 7.9, at least 8.0, at least 8.1, at least 8.2, at least 8.3, at least 8.4, at least 8.5, at least 8.6, at least 8.7, at least 8.8, at least 8.9, or at least 9.0. The pH may be about 7.0, about 7.1, about 7.2, about 7.3, about 7.4, about 7.5, about 7.6, about 7.7, about 7.8, about 7.9, about 8.0, about 8.1, about 8.2, at least 8.3, about 8.4, about 8.5, about 8.6, about 8.7, about 8.8, about 8.9, or about 9.0.

In one embodiment, provided herein are kits comprising the compositions of any of the present embodiments. The kits may further comprise a solid silica substrate, such as silica beads, silica-coated beads, a silica column, or a silica membrane. The silica-coated beads may be magnetic or paramagnetic.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—cfDNA extraction without proteinase K using EDTA or MGDA and sodium percarbonate in the binding buffer.

FIG. 2—cfDNA extraction without proteinase K using sodium triphosphate and sodium percarbonate or hydrogen peroxide in the binding buffer.

FIG. 3—cfDNA extraction without proteinase K using either a chelating agent or sodium percarbonate in the binding buffer.

FIG. 4—cfDNA extraction using a commercial kit modified to exclude the proteinase K step and to include EDTA and sodium percarbonate in the binding buffer.

FIG. 5—cfDNA extraction without proteinase K using EDTA and sodium percarbonate in the wash buffer.

### DETAILED DESCRIPTION

One commonly used method for extraction of nucleic acids from biological samples involves the addition of a chaotropic agent to a complex biological sample to form a lysate, followed by binding of nucleic acids to a solid silica substrate (U.S. Pat. No. 5,234,809). Under some conditions, the addition of alcohol to the lysate may improve nucleic acid binding to the silica substrate. After binding of the nucleic acids to the silica substrate and removal of the non-bound components, one or more washes are performed, followed by elution of the nucleic acids from the substrate. The silica substrate may be provided in the form of a packed column (stationary solid phase) or in the form of particles (mobile solid phase), which may have magnetic or paramagnetic cores to facilitate separation from the non-binding components of the lysate.

Efficient isolation of nucleic acids from blood products using the aforementioned method is known to be challenging. In particular, blood products tend to plug stationary solid phase systems, preventing the lysate from passing through the solid phase. Use of blood products with mobile solid phase systems generally result in poor yields of nucleic acids. To overcome this problem, blood product lysates are typically treated with a proteinase K to remove unwanted protein prior to addition to silica substrates. However, such treatments add to the cost and complexity of nucleic acid extraction, as they often require prolonged heat treatment and separate addition of proteinase K and chaotropic agents and alcohol.

In recent years, interest in analysis of circulating or cell-free DNA (cfDNA) has grown. By way of example, cfDNA can be analyzed for the diagnosis and prognosis, as well as the early detection, of cancer; pre-natal diagnosis of fetal genetic conditions; detection of biomarkers of myocardial infarction or transplant graft rejection. cfDNA is extracted from blood plasma, and various methods have been developed to optimize extraction of these extracellular nucleic acids (see, e.g., Jorgez et al., Genet. Med. (2006) 8:615-619; U.S. Pat. Publn. US2017/0183712; U.S. Pat. Publn. US2019/0225958). cfDNA exists within nucleosomes and efficient extraction for subsequent analysis is dependent on releasing the nucleic acids from the protein components of the nucleosomes. One well established commercially available method makes use of the QIAamp® Circulating Nucleic Acid Kit (Qiagen N.V.), which includes a pretreatment with proteinase K prior to addition of the lysate to the solid silica matrix.

The compositions and methods disclosed herein eliminate the need for pretreatment with proteases prior to binding of nucleic acids to silica substrates. In one embodiment, plasma containing nucleic acids of interest is added to a solution containing a high concentration of a chaotropic agent in the presence of a chelating agent (e.g., EDTA, MGDA, or sodium triphosphate) and sodium percarbonate. In another embodiment, nucleic acids bound to a solid substrate are washed with a solution containing a chelating agent (e.g., EDTA, MGDA, or sodium triphosphate) and sodium percarbonate. In alternate embodiments, sodium percarbonate is replaced by hydrogen peroxide in the presence of sodium triphosphate. By eliminating the requirement for pretreating the biological samples with proteinase K prior to binding nucleic acids to the silica substrate, the complexity, cost, and time taken to extract nucleic acids for further analysis is reduced. The disclosed composition and methods also facilitate elution without the need for heat as well as the addition of alcohol (e.g., isopropyl alcohol) to the binding buffer prior to use in the extraction with equivalent results.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

By “biological sample” is meant a sample comprising any biological material from which samples can be prepared for use in the methods disclosed herein. This includes, but is not limited to, blood, plasma, serum, saliva, urine, cerebral spinal fluid, pleural fluid, milk, lymph, sputum, and semen.

By “chelator” or “chelating agent” is meant chemical compounds that react with metal ions to form a stable, water-soluble complex. The chelator may be an amine-based chelator or a phosphate-based chelator. The chelator may be ethylenediaminetetraacetic acid (“EDTA”), ethylene glycol-bis(2-aminoethylether) tetraacetic acid (“EGTA”), methylglycinediacetic acid (“MGDA”), sodium triphosphate (“STP”), nitrilotriacetic acid (“NTA”), iminodisuccinic acid (“IDS”), polyaspartic acid, S,S-ethylenediamine-N,N′-disuccinic acid (“EDDS”), L-Glutamic acid N,N-diacetic acid, tetrasodium salt (“GLDA”), or their salts. The terms “EDTA,” “MGDA,” etc. will be used to refer both to the acid and the salt form, and either form may be used in the present invention.

By “oxidizing agent” is meant a substance that accepts electrons from another substance and is thus itself reduced. Exemplary oxidizing agents include peroxide-containing compounds, such as, for example, hydrogen peroxide, sodium percarbonate, urea peroxide, melamine peroxide, potassium percarbonate, sodium perborate, potassium perborate, sodium peroxide, potassium peroxide, magnesium peroxide, barium peroxide, calcium peroxide, strontium peroxide, hydrogen peroxide adducts of sulfates, hydrogen peroxide adducts of phosphates, hydrogen peroxide adducts of pyrophosphates, and the like

By “chaotropic agent” is meant agents that disrupt molecular structure, particularly molecular structure formed by nonbonding forces such as hydrogen bonding, Van der Waals interaction, and hydrophobic effect. Chaotropic agents are well known in the field of biochemistry and include, but are not limited to, guanidine hydrochloride, guanidine thiocyanate, guanidine isothiocyanate, sodium perchlorate, sodium iodide, sodium trichloroacetate, urea, thiourea, rhodanite salt, aminoguanidine bicarbonate, guanidine carbonate, guanidine phosphate, and aminoguanidine hydrochloride.

By “ambient temperature” is meant room temperature and generally refers to a temperature that is between about 20° C. and about 30° C. (e.g., about 20° C., 21° C., 22° C., 23° C., 24° C., 25° C., 26° C., 27° C., 28° C., 29° C., or 30° C.).

As used herein “nucleic acid” means either DNA or RNA, either single-stranded or double-stranded.

As used herein, “amplification” or “amplifying” refers to the in vitro production of additional copies of a target nucleic acid sequence. Amplification is generally carried out using polymerase chain reaction (PCR) technologies known in the art. The term “amplification reaction” refers to an aqueous solution comprising the various reagents used to amplify a target nucleic acid. These may include enzymes (e.g., a thermostable polymerase), aqueous buffers, salts, amplification primers, target nucleic acid, nucleoside triphosphates, and optionally, at least one labeled probe and/or optionally, at least one agent for determining the melting temperature of an amplified target nucleic acid (e.g., a fluorescent intercalating agent that exhibits a change in fluorescence in the presence of double-stranded nucleic acid).

The term “PCR” or “polymerase chain reaction” encompasses derivative forms of the reaction, including but not limited to, RT-PCR, real-time PCR, nested PCR, quantitative PCR, multiplexed PCR, assembly PCR, digital PCR, and the like. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., U.S. Pat. No. 5,210,015 (“Taqman”); U.S. Pat. Nos. 6,174,670 and 6,569,627 (intercalating dyes); U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. “Initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture. Usually, distinct sets of primers are employed for each sequence being amplified. “Quantitative PCR” means a PCR designed to measure the abundance of one or more specific target sequences in a sample or specimen. “Digital PCR” involves partitioning the sample such that individual nucleic acid molecules contained in the sample are localized in many separate regions, such as in individual wells in microwell plates, in the dispersed phase of an emulsion, or arrays of nucleic acid binding surfaces. Each partition will contain 0 or 1 molecule, providing a negative or positive reaction, respectively.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Plasma Preparation and Buffer Preparation

In order to prepare plasma, blood was drawn into EDTA-containing tubes and centrifuged at 1900 g for 10 min to sediment cellular material. The supernatant containing the plasma was collected and recentrifuged at 6000 g for 10 min. Then, the supernatant plasma was collected and stored at −80° C. until needed.

Modified Binding Buffer 1 (MBB1) was prepared as follows: 3.4 M guanidine, 37 mM TRIS, 2.5% Tween 20, 2.3% Triton X-100, 3.8% Simethicone Emulsion, 20 mM EDTA, 40 mM sodium percarbonate, 22% isopropyl alcohol (IPA). The solution was stored for future use at ambient temperature protected from light.

Modified Binding Buffer 2 (MBB2) was prepared as follows: 3.4 M guanidine, 37 mM TRIS, 2.5% Tween 20, 2.3% Triton X-100, 3.8% Simethicone Emulsion, 11 mM MGDA (methylglycinediacetic acid trisodium salt), 40 mM sodium percarbonate, 22% isopropyl alcohol (IPA). The solution was stored for future use at ambient temperature protected from light.

Modified Binding Buffer 3 (MBB3) was prepared as follows: 3.4 M guanidine, 37 mM TRIS, 2.5% Tween 20, 2.3% Triton X-100, 3.8% Simethicone Emulsion, 14 mM STP (sodium triphosphate), 40 mM sodium percarbonate, 22% isopropyl alcohol (IPA). The solution was stored for future use at ambient temperature protected from light.

Modified Binding Buffer 4 (MBB4) was prepared as follows: 3.4 M guanidine, 37 mM TRIS, 2.5% Tween 20, 2.3% Triton X-100, 3.8% Simethicone Emulsion, 14 mM STP (sodium triphosphate), 5 mM hydrogen peroxide, 22% isopropyl alcohol (IPA). The solution was stored for future use at ambient temperature protected from light.

### Example 2—cfDNA Extraction Using EDTA or MGDA and Sodium Percarbonate

Cell-free DNA (cfDNA) was extracted from human plasma using methods disclosed herein, and the extracted cfDNA was used for further analysis by PCR. Results were compared to those obtained using standard methods of extraction with and without pretreatment using proteinase K and to those obtained using a commercially available kit for cfDNA extraction (QIAamp®, obtained from Qiagen N.V.).

Extraction using the standard mobile solid phase (Standard) method was performed as follows. First, 1 mL of human plasma was obtained as described in Example 1, added to 500 μL proteinase K (QIAGEN; Cat. No. 19131), and incubated at 60° C. for 30 min. Then, 2.5 mL of standard binding buffer (4.5 M guanidine, 50 mM TRIS, 3.3% Tween 20, 3% Triton X-100, 5% Simethicone Emulsion), 750 μL isopropyl alcohol (IPA), and 250 μL magnetic silica particles (Luminex Corp.) were added to the incubated plasma, and then the mixture was vortexed. The mixture was incubated at ambient temperature for at least 10 min, and the particles were collected on the side of the tube to permit removal of the liquid. Particles were washed once with Wash Buffer 1 (0.5 M guanidine, 2.5% Triton X-100, 2.5% Tween 20, 50% IPA, 100 mM 2-(N-morpholino)ethanesulfonic acid (MES), 2 mM EDTA) and once with Wash Buffer 2 (pH 5.5, 20 mM MES). Nucleic acids were eluted from the particles in 50 μL, Elution Buffer (10 mM Tris, pH 8.1) at 70° C. for 10 min on an Eppendorf ThermoMixer® at 1000 rpm.

Extraction using the Standard method without pretreatment with proteinase K was performed by omitting the addition of proteinase K and the 60° C. incubation.

Extraction using the QIAamp® kit was performed in accordance with the manufacturer's instructions.

Extraction using the disclosed MBB1 and MBB2 methods was performed as follows. First, 1 mL of human plasma was obtained as described in Example 1 and added to 3.250 mL Modified Binding Buffer 1 (MBB1) or Modified Binding Buffer 2 (MBB2). After briefly vortexing, 100 μL, of magnetic silica particles, as described above, were added to the mixture, which was vortexed to resuspend the particles. After a 7-minute incubation at ambient temperature, particles were collected on the side of the tube to permit removal of the liquid. Particles were washed once with Wash Buffer 1 (0.5 M guanidine, 2.5% Triton X-100, 2.5% Tween 20, 50% IPA, 100 mM 2-(N-morpholino)ethanesulfonic acid (MES), 2 mM EDTA) and once with Wash Buffer 2 (pH 5.5, 20 mM MES). Nucleic acids were eluted from the particles by vortexing to resuspend the magnetic particles in 50 μL, Elution Buffer (10 mM Tris, pH 8.1) and incubating at ambient temperature for 30 sec.

PCR analysis of the extracted nucleic acids was performed as follows. A TAQMAN® assay using the RPP30 human reference gene as a target was used to evaluate suitability of extracted nucleic acid for subsequent analysis. Cₜ values from qPCR of nucleic acids extracted using the various methods were compared. Eluted nucleic acid (5 μL) was added to 25 μL, PCR Master Mix (PHOENIX™ Taq DNA Polymerase (QIAGEN) 0.08 U/μL; 10 mM Bis-Tris propane, 90 nM DTT, 6 mg/mL BSA, 10 mM dTTP, 10 mM dGTP, 10 mM dCTP, 10 mM dATP, 10 nM Tris, 2.5 nM MgCl₂, 50 nM KCl). Primers and probe were added (200 nM Forward: 5′-GATTTGGACCTGCGAGCG-3′; 200 nM Reverse: 5′-GCGGCTGTCTCCACAAGT-3′; 400 nM Probe: 5′-VIC-CTGACCTGAAGGCTCT-3′). PCR reactions were cycled on an ABI 7500 instrument as follows: 140 sec at 95° C., followed by 45 cycles of 10 sec at 95° C. and 30 sec at 58° C. Fluorescence was measured in the VIC channel set up for NFQ-MGB quench.

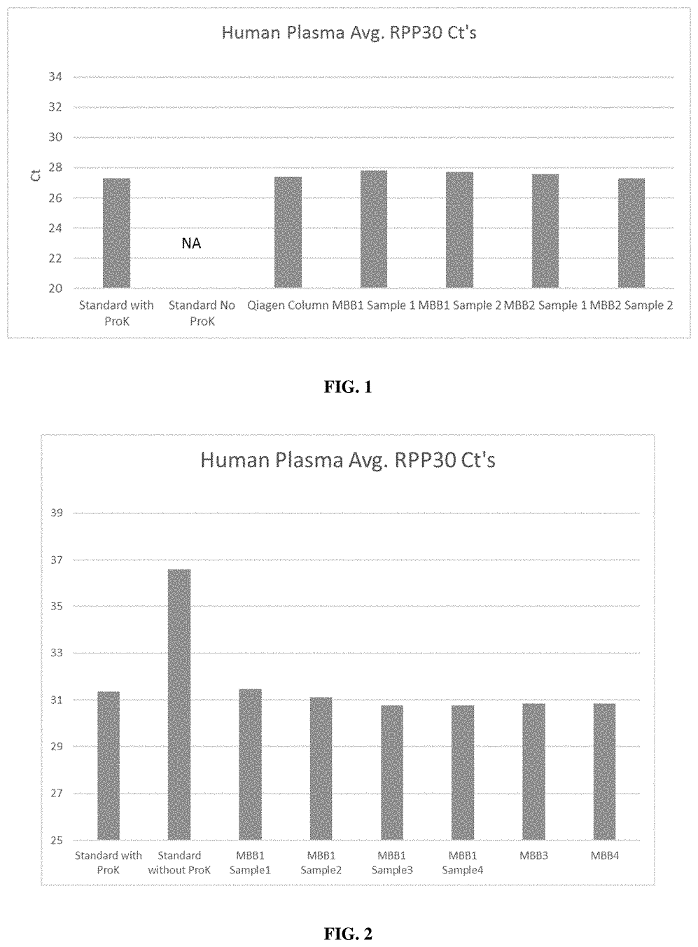

cfDNA extracted using the disclosed MBB1 and MBB2 methods yielded similar Cₜ values to the Standard method with proteinase K treatment or the kit (FIG. 1). No signal was detected from cfDNA extracted using the Standard method without pretreatment with proteinase K, indicating that the disclosed MBB1 and MBB2 methods rendered this step unnecessary.

### Example 3—cfDNA Extraction Using Sodium Triphosphate and Sodium Percarbonate or Hydrogen Peroxide

cfDNA was extracted from human plasma using the MBB1 and MBB2 methods described in Example 2, except that Modified Binding Buffer 3 (MBB3) and Modified Binding Buffer 4 (MBB4) were used. Results were compared to those obtained using the Standard method, the Standard method without pretreatment with proteinase K, and the MBB1 method, each described in Example 2.

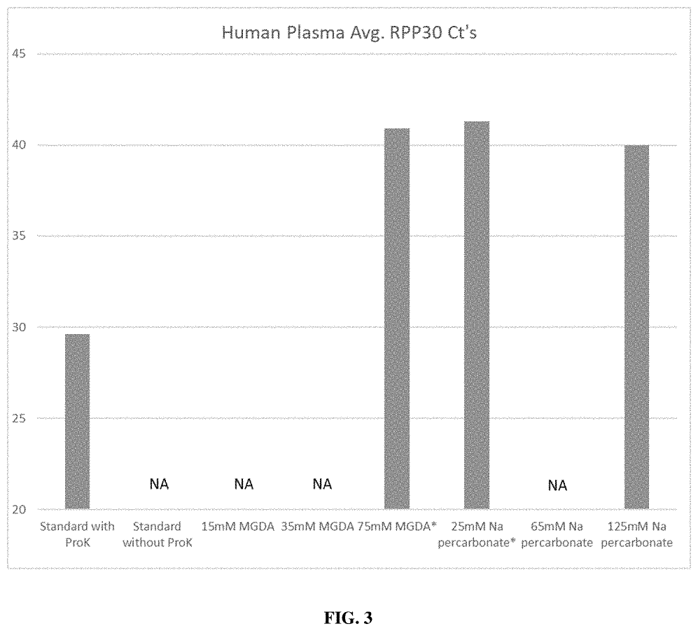

Note that 200 μL of plasma was used in these experiments and other reagents were adjusted accordingly to maintain the same relative concentrations as described in Example 2. The results are shown in FIG. 2 and demonstrate that MBB3 and MBB4 performed equivalently to MBB1, and all Modified Binding Buffer experiments showed similar results to the Standard method with proteinase K treatment. Once again, the Standard method that did not include a proteinase K pretreatment step yielded much less desirable results, indicating the need for proteinase K pretreatment in the Standard method. These results confirm that the methods disclosed here, which use a phosphate-based chelating agent (trisodium phosphate), provide comparable quantitative amplification results to amine-based chelating agents, such as EDTA. In addition, hydrogen peroxide provides quantitative PCR results comparable to sodium percarbonate when used with trisodium phosphate.

### Example 4—cfDNA Extraction Using Either a Chelating Agent or Sodium Percarbonate

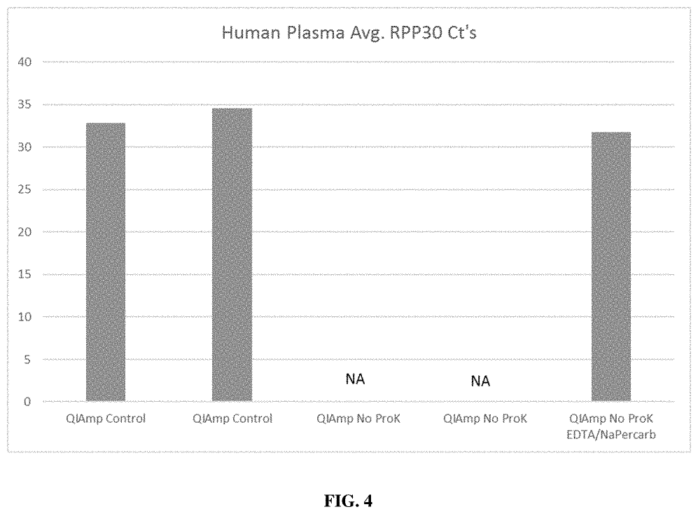

Aliquots of plasma (200 μL) were processed (a) using the Standard method without pretreatment with proteinase K, as described in Example 2, (b) using the QIAamp® kit in accordance with the manufacturer's instructions, (c) using the Standard method without pretreatment with proteinase K, except that the standard binding buffer contained 15, 35, or 75 mM MGDA, and (d) using the Standard method without pretreatment with proteinase K, except that the standard binding buffer contained 25, 65, or 125 mM sodium percarbonate. Results are shown in FIG. 3 below and indicate that use of either a chelating agent alone or sodium percarbonate alone do not improve extraction of cfDNA from plasma without proteinase K pretreatment.

### Example 5—Addition of a Chelating Agent and Sodium Percarbonate in a Commercial Kit

cfDNA was extracted from human plasma using the QIAamp® cfDNA kit protocol. One sample was processed according to the manufacturer's protocol. A second sample was processed according to the same protocol, but with the proteinase K step omitted. A third sample was also processed without the proteinase K step, but with the buffer provided in the kit modified to include a chelating agent (20 mM EDTA) and sodium percarbonate (40 mM).

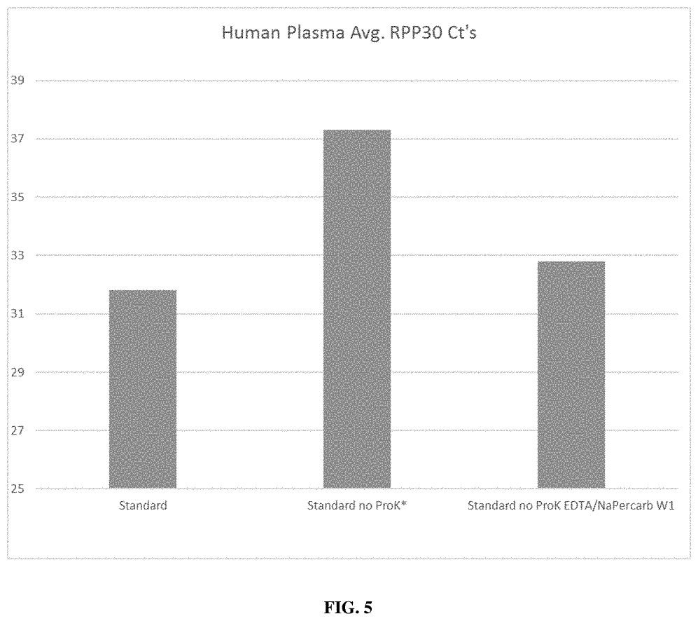

Omitting the proteinase K step resulted in no amplifiable nucleic acid being recovered (FIG. 4). However, omitting the proteinase K step and modifying the kit-provided buffer by addition of a chelating agent and sodium percarbonate yielded a similar quantitative result to the manufacturer's protocol, which includes a proteinase K step (FIG. 4).

### Example 6—Extraction of cfDNA Using Modified Wash Buffer

Nucleic acid was extracted from 200 μL aliquots of plasma collected and prepared as described in Example 1. A first aliquot was processed using the Standard method with proteinase K pretreatment as described in Example 2. A second aliquot was processed using the Standard method without proteinase K pretreatment as described in Example 2. A third aliquot was processed using the Standard method without proteinase K pretreatment, but with Wash Buffer 1 modified to include chelating agent (25 mM EDTA) and sodium percarbonate (50 mM). Note that for all three aliquots, remaining reagent volumes as provided in Example 2 were adjusted to account for the reduced sample volume.

The addition of chelating agent and sodium percarbonate to the Wash Buffer 1 significantly improved the efficiency of extraction of cfDNA when compared to the Standard method without a proteinase K pretreatment step (FIG. 5). Without wishing to be bound by theory, the inventors believe that the improved efficiency of nucleic acid amplification (as evidenced by lower Cₜ values) when sodium percarbonate and chelating agent was added to either binding buffer or wash buffer was the result of improved elution of bound nucleic acids from the solid support.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 5,210,015

U.S. Pat. No. 5,234,809

U.S. Pat. No. 5,925,517

U.S. Pat. No. 6,174,670

U.S. Pat. No. 6,569,627

U.S. Pat. Publn. US2017/0183712

U.S. Pat. Publn. US2019/0225958

Jorgez et al., Genet. Med., 8:615-619 (2006)

Mackay et al., Nucleic Acids Research, 30:1292-1305 (2002)

1. A method for extracting cell-free DNA (cfDNA) from a biological sample, the method comprising:
(1) combining the biological sample with a solid substrate and a binding buffer comprising a chaotropic agent in order to bind cfDNA from the biological sample to the solid substrate, wherein the biological sample has not been treated with proteinase K;
(2) washing the cfDNA bound to the solid substrate with a wash buffer; and
(3) eluting the cfDNA from the solid substrate,
wherein the binding buffer and/or the wash buffer comprises a chelating agent and an oxidizing agent.

2. The method of claim 1, wherein the binding buffer comprises a chaotropic agent, a chelating agent, and an oxidizing agent.

3. The method of claim 1, wherein the wash buffer comprises a chelating agent and an oxidizing agent.

4. The method of claim 1, wherein eluting is performed at ambient temperature.

5. The method of claim 1, wherein the method does not include a heat treatment step.

6. The method of claim 1, wherein the chelating agent is an amine-based chelating agent or a phosphate-based chelating agent.

7. The method of claim 1, wherein the oxidizing agent is sodium percarbonate or hydrogen peroxide.

8. The method of claim 1, wherein the chelating agent is EDTA or MGDA and the oxidizing agent is sodium percarbonate.

9. The method of claim 1, wherein the chelating agent is STP and the oxidizing agent is sodium percarbonate or hydrogen peroxide.

10. The method of claim 1, wherein the binding buffer comprises an alcohol.

11. The method of claim 1, wherein the binding buffer comprises isopropyl alcohol.

12. The method of claim 1, wherein the solid substrate is a silica substrate.

13. The method of claim 1, wherein the solid substrate is silica beads, silica-coated beads, a silica column, or a silica membrane.

14. The method of claim 1, wherein the biological sample is a blood plasma sample.

15-33. (canceled)
